(19) United States
(12) Patent Application Publication
(10) Pub. No.: US 2014/0128716 A1
(43) Pub. Date: May 8, 2014

(54) METHOD AND SYSTEM FOR SENSING RADIO WAVE IN DIAGNOSTIC IMAGING OF BREAST CANCER

(71) Applicant: Electronics and Telecommunications Research Institute, Daejeon (KR)

(72) Inventors: Seong Ho SON, Daejeon (KR); Soon Ik JEON, Daejeon (KR); Nikolai SIMONOV, Daejeon (KR); Jong Moon LEE, Chungcheongbuk-do (KR); Hyuk Je KIM, Daejeon (KR); Hyung Do CHOI, Seoul (KR)

(21) Appl. No.: 13/912,465

(22) Filed: Jun. 7, 2013

(30) Foreign Application Priority Data
Nov. 5, 2012 (KR) 10-2012-0123943

(51) Int. Cl. A61B 5/05; A61B 5/00

(52) U.S. Cl. CPC A61B 5/05; A61B 5/7235
USPC 600/407

(57) ABSTRACT

Provided is a method of sensing a radio wave in diagnostic imaging of breast cancer, including arranging a plurality of transmitting and receiving antennas along a periphery of a breast, transmitting a radio wave signal from at least one first antenna among the plurality of transmitting and receiving antennas, receiving a scattered wave signal for the radio wave signal from the remaining antennas, other than the first antenna, among the plurality of transmitting and receiving antennas, and rotating the plurality of transmitting and receiving antennas by a predetermined angle.

### CROSS-REFERENCE TO RELATED APPLICATION

This application claims the priority benefit of Korean Patent Application No. 10-2012-0123943, filed on Nov. 5, 2012, in the Korean Intellectual Property Office, the disclosure of which is incorporated herein by reference.

### BACKGROUND

1. Field of the Invention

The present invention relates to a system and method for sensing a radio wave in diagnostic imaging of breast cancer, and more particularly, to a technique that uses a transmitting and receiving antenna in radio wave sensing for diagnostic imaging of breast cancer.

2. Description of the Related Art

In the radio wave sensing for diagnostic imaging of breast cancer, a technique that uses a transmitting and receiving antenna involves transmitting and receiving a radio wave signal using transmitting and receiving antennas arranged around a breast of a subject, analyzing a scattered wave signal for the received radio wave signal, and reconstructing a tomographic image of the breast of the subject for breast cancer diagnosis.

### SUMMARY

An aspect of the present invention provides, in diagnostic imaging of breast cancer, a method, apparatus, and system for high precision sensing of a radio wave along a periphery of a breast using a plurality of transmitting and receiving antennas.

Another aspect of the present invention also provides, in diagnostic imaging of breast cancer, a method, apparatus, and system for high precision sensing of a radio wave by rotating and moving a plurality of transmitting and receiving antennas in the process of using the plurality of transmitting and receiving antennas.

Still another aspect of the present invention also provides, in diagnostic imaging of breast cancer, a method, apparatus, and system for high precision sensing of a radio wave in a depthwise direction of a breast using a plurality of transmitting and receiving antennas for a plurality of layers.

Yet another aspect of the present invention also provides a method, apparatus, and system for arranging a plurality of transmitting and receiving antennas in each of a plurality of layers of a breast in the process of using the plurality of transmitting and receiving antennas for the plurality of layers.

According to an aspect of the present invention, there is provided a method of sensing a radio wave in diagnostic imaging of breast cancer, the method including arranging a plurality of transmitting and receiving antennas along a periphery of a breast, transmitting a radio wave signal from at least one first antenna among the plurality of transmitting and receiving antennas, receiving a scattered wave signal for the radio wave signal from the remaining antennas, other than the first antenna, among the plurality of transmitting and receiving antennas, and rotating the plurality of transmitting and receiving antennas by a predetermined angle.

The rotating of the plurality of transmitting and receiving antennas may include re-arranging the plurality of transmitting and receiving antennas by rotating the plurality of transmitting and receiving antennas by the predetermined angle along the periphery of the breast.

The method may further include moving the plurality of transmitting and receiving antennas by a predetermined distance in a depthwise direction of the breast after rotating the plurality of transmitting and receiving antennas.

The method may further include setting at least one new first antenna among the remaining antennas, other than the first antenna transmitting the radio wave signal, after receiving the scattered wave signal.

The receiving of the scattered wave signal for the radio wave signal may include obtaining radio wave signal amplitude and phase information from the received radio wave signal.

According to another aspect of the present invention, there is provided a method of sensing a radio wave in diagnostic imaging of breast cancer, the method including arranging a plurality of transmitting and receiving antennas along a periphery of a breast in a plurality of layers in a depthwise direction of the breast, transmitting a radio wave signal from at least one first antenna among the plurality of transmitting and receiving antennas for the plurality of layers, receiving a scattered wave signal for the radio wave signal from the remaining antennas other than the first antenna among the plurality of transmitting and receiving antennas for the plurality of layers, and moving the plurality of transmitting and receiving antennas for the plurality of layers by a predetermined distance in the depthwise direction of the breast.

The arranging of the plurality of transmitting and receiving antennas in the plurality of layers may include arranging the plurality of transmitting and receiving antennas in the plurality of layers in a staggered manner, relative to the transmitting and receiving antennas of adjacent layers.

The method may further include setting at least one new first antenna among the remaining antennas, other than the first antenna transmitting the radio wave signal, after receiving the scattered wave signal in the plurality of layers.

The method may further include transmitting the radio wave signal from the at least one first antenna among the plurality of transmitting and receiving antennas for preset adjacent layers among the plurality of layers, and receiving the scattered wave signal for the radio wave signal from the remaining antennas, other than the first antenna, among the plurality of transmitting and receiving antennas for the preset adjacent layers.

The method may further include setting at least one new first antenna among the remaining antennas, other than the first antenna transmitting the radio wave signal, after receiving the scattered wave signal in the preset adjacent layers.

According to still another aspect of the present invention, there is provided a system for sensing a radio wave in diagnostic imaging of breast cancer, the system including a plurality of transmitting and receiving antennas, a rotation unit, and a movement unit, the plurality of transmitting and receiving antennas may be arranged along a periphery of a breast, at least one first antenna among the plurality of transmitting and receiving antennas may transmit a radio wave signal, and the remaining antennas, other than the first antenna, may receive a scattered wave signal for the radio wave signal, and the rotation unit may rotate the plurality of transmitting and receiving antennas by a predetermined angle and re-arrange the plurality of transmitting and receiving antennas by rotating the plurality of transmitting and receiving antennas by the predetermined angle along the periphery of the breast.

The movement unit may move the plurality of transmitting and receiving antennas by a predetermined distance in a depthwise direction of the breast after the plurality of transmitting and receiving antennas are rotated, and after the scattered wave signal is received from the remaining antennas, other than the first antenna, at least one new first antenna may be set among the remaining antennas, other than the first antenna transmitting the radio wave signal.

According to yet another aspect of the present invention, there is provided a system for sensing a radio wave in diagnostic imaging of breast cancer, the system including a plurality of transmitting and receiving antennas for a plurality of layers, and a movement unit, the plurality of transmitting and receiving antennas for the plurality of layers may be arranged along a periphery of a breast in the plurality of layers in a depthwise direction of the breast, at least one first antenna among the plurality of transmitting and receiving antennas for the plurality of layers may transmit a radio wave signal, and the remaining antennas, other than the first antenna, among the plurality of transmitting and receiving antennas for the plurality of layers may receive a scattered wave signal for the radio wave signal, and the movement unit may move the plurality of transmitting and receiving antennas for the plurality of layers by a predetermined distance in the depthwise direction of the breast.

The plurality of transmitting and receiving antennas for the plurality of layers may be arranged in a staggered manner, relative to the transmitting and receiving antennas of adjacent layers, and after the scattered wave signal is received from the remaining antennas, other than the first antenna, among the plurality of transmitting and receiving antennas for the plurality of layers, at least one new first antenna may be set among the remaining antennas, other than the first antenna transmitting the radio wave signal.

The at least one first antenna among the plurality of transmitting and receiving antennas for preset adjacent layers among the plurality of layers may transmit the radio wave signal, and the remaining antennas, other than the first antenna, among the plurality of transmitting and receiving antennas for the preset adjacent layers may receive a scattered wave signal for the radio wave signal.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a diagram illustrating radio wave sensing along a periphery of a breast in diagnostic imaging of breast cancer according to an exemplary embodiment;

FIG. 2 is a diagram specifically illustrating radio wave sensing along a periphery of a breast in diagnostic imaging of breast cancer according to an exemplary embodiment;

FIG. 3 is a diagram illustrating radio wave sensing in a depthwise direction of a breast in diagnostic imaging of breast cancer according to an exemplary embodiment;

FIG. 4 is a flowchart illustrating a method of sensing a radio wave along a periphery of a breast in diagnostic imaging of breast cancer according to an exemplary embodiment;

FIG. 5 is a flowchart illustrating a method of sensing a radio wave in a depthwise direction of a breast in diagnostic imaging of breast cancer according to an exemplary embodiment;

FIG. 6 is a block diagram illustrating a system for sensing a radio wave along a periphery of a breast in diagnostic imaging of breast cancer according to an exemplary embodiment; and

FIG. 7 is a block diagram illustrating a system for sensing a radio wave in a depthwise direction of a breast in diagnostic imaging of breast cancer according to an exemplary embodiment.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

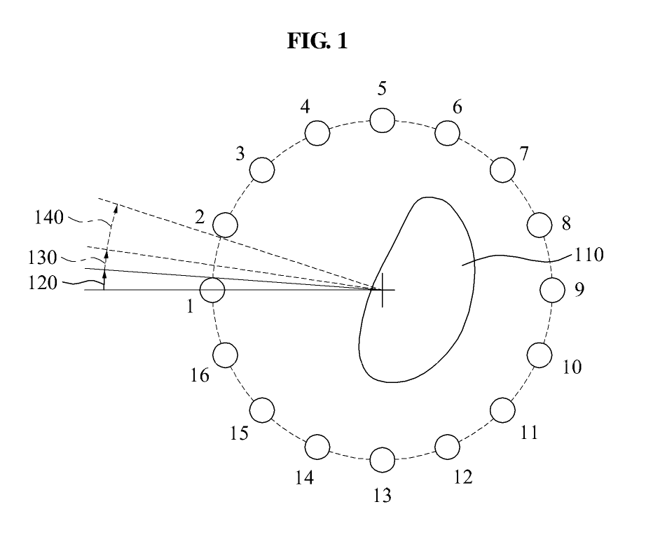

FIG. 1 illustrates radio wave sensing along a periphery of a breast in diagnostic imaging of breast cancer according to an exemplary embodiment.

Referring to FIG. 1, radio wave sensing for diagnostic imaging of breast cancer according to an exemplary embodiment may include a breast 110 and a plurality of antennas, for example, antenna numbers (Nos.) 1 through 16. Here, the plurality of antennas may correspond to transmitting and receiving antennas arranged in a circular form. The breast 110 of a subject may be inserted in between the plurality of antennas.

A system according to an exemplary embodiment may execute radio wave sensing by setting a first antenna among the plurality of antennas, transmitting a radio wave signal from the first antenna, and receiving a scattered wave signal for the radio wave signal from the remaining antennas, other than the first antenna. Here, the receiving antennas may obtain radio wave signal amplitude and phase information from the received radio wave signal.

In this instance, after the scattered wave signal for the radio wave signal transmitted from the first antenna is received from the remaining antennas, other than the first antenna, and the system according to an exemplary embodiment may set a new first antenna among the remaining antennas, other than the first antenna transmitting the radio wave signal. The new first antenna may transmit a radio wave signal, and the remaining antennas, other than the new first antenna, may receive a scattered wave signal for the radio wave signal.

This process may be performed iteratively until the plurality of antennas, for example, antenna Nos. 1 through 16 are all set to be a first antenna.

The system according to an exemplary embodiment may rotate the plurality of antennas by a predetermined angle, for example, an angle 120, an angle 130, and an angle 140, along a periphery of the breast 110. When radio wave sensing is completed for each of the plurality of antennas by setting a first antenna among the plurality of antennas, transmitting a radio wave signal from the first antenna, and receiving a scattered wave signal for the radio wave signal from the remaining antennas, other than the first antenna, the plurality of antennas may be rotated by the angle 120 along the periphery of the breast 110.

When the plurality of antennas are rotated by the angle 120, the radio wave sensing may be executed again, and when the plurality of antennas are rotated by the angle 130, the radio wave sensing may be executed again. Subsequently, the plurality of antennas may be rotated by the angle 140.

After the plurality of antennas are rotated, the system according to an exemplary embodiment may move the plurality of antennas by a predetermined distance in a depthwise direction of the breast 110, and may execute the radio wave sensing again as described in the foregoing.

Hereinafter, a further detailed description is provided.

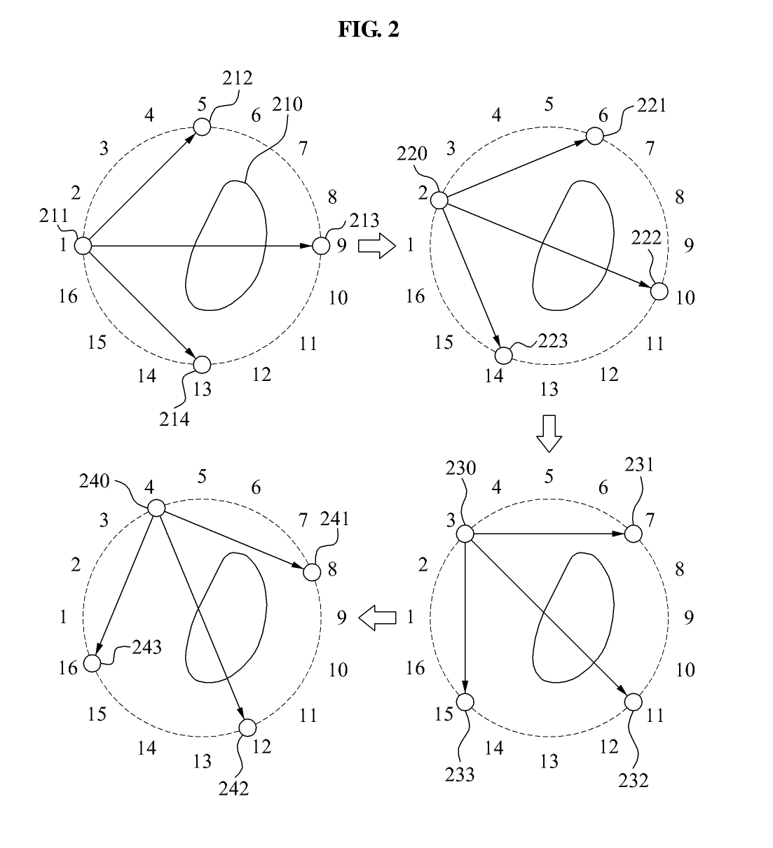

FIG. 2 specifically illustrates radio wave sensing along a periphery of a breast in diagnostic imaging of breast cancer according to an exemplary embodiment.

Referring to FIG. 2, radio wave sensing in diagnostic imaging of breast cancer according to an exemplary embodiment may include a breast 210 and a plurality of antennas, for example, antenna Nos. 1 through 4. Here, a system according to an exemplary embodiment may execute radio wave sensing using an antenna No. 1 211, 220, 230, and 240, an antenna No. 2 212, 221, 231, and 241, an antenna No. 3 213, 222, 232, and 242, and an antenna No. 4 214, 223, 233, and 243, at a plurality of locations, for example, location Nos. 1 through 16.

The system according to an exemplary embodiment may arrange the plurality of antennas in a first arrangement. More particularly, the system may place the antenna No. 1 211 at a location No. 1 and the antenna No. 2 212 at a location No. 5 along a periphery of the breast 210. Also, the system may place the antenna No. 3 213 at a location No. 9 and the antenna No. 4 214 at a location No. 13 along the periphery of the breast 210.

In this instance, the antenna No. 1 211 may be set to be a first antenna and may transmit a radio wave signal to the remaining antennas 212, 213, and 214, other than the first antenna 211, and the remaining antennas 212, 213, and 214 may receive a scattered wave signal for the radio wave signal. Also, the antenna No. 2 212 may be set to be a new first antenna and may transmit a radio wave signal, and the remaining antennas 211, 213, and 214, other than the first antenna 212, may receive a scattered wave signal for the radio wave signal. In the same manner as the antenna No. 1 211 and the antenna No. 212, the antenna No. 213 and the antenna No. 4 214 may perform a series of operations. More particularly, the antenna No. 4 214 corresponding to a last antenna among the plurality of antennas 211, 212, 213, and 214 may be set to be a new first antenna and may transmit a radio wave signal, and the remaining antennas 211, 212, and 213, other than the first antenna 214, may receive a scattered wave signal for the radio wave signal. Transitively, this process may be performed iteratively until the plurality of antennas, for example, antennas Nos. 1 through 4 are all set to be a first antenna in the first arrangement.

After radio wave sensing is completed in the first arrangement, the plurality of antennas may be rotated by a predetermined angle along the periphery of the breast 210. As a result of the rotation, the plurality of antennas 220, 221, 222, and 223 may be in a second arrangement. More particularly, an antenna No. 1 220 may be placed at a location No. 2, and an antenna No. 2 221 may be placed at a location No. 6. Also, an antenna No. 3 222 may be placed at a location No. 10, and an antenna No. 4 223 may be placed at a location No. 14.

Similarly, radio wave sensing may be proceeded with by setting the antenna No. 1 220 to be a first antenna, and after the radio wave sensing is completed in the second arrangement, the plurality of antennas 220, 221, 222, and 223 may be rotated by a predetermined angle along the periphery of the breast 210.

The plurality of antennas may be re-arranged in a third arrangement. More particularly, an antenna No. 1 230 may be placed at a location No. 3, and an antenna No. 2 231 may be placed at a location No. 7. Also, an antenna No. 3 232 may be placed at a location No. 11, and an antenna No. 4 233 may be placed at a location No. 15. In the third arrangement, radio wave sensing may be executed by setting a first antenna among the plurality of antennas, transmitting a radio wave signal from the first antenna, and receiving a scattered wave signal for the radio wave signal from the remaining antennas, other than the first antenna. This process may be performed iteratively until the plurality of antennas, for example, antenna Nos. 1 through 4 are all set to be a first antenna.

Also, the plurality of antennas may be re-arranged by being rotated a predetermined angle the periphery of the breast 210 from the third arrangement. The plurality of antennas 240, 241, 242, and 243 may be in a fourth arrangement. More particularly, an antenna No. 1 240 may be placed at a location No. 4, an antenna No. 2 241 may be placed at a location No. 8, an antenna No. 3 242 may be placed at a location No. 12, and an antenna No. 4 243 may be placed at a location No. 16. In this instance, radio wave sensing may be executed in the fourth arrangement as described in the foregoing.

Also, after the plurality of antennas, for example, antennas Nos. 1 through 4 are rotated, the system according to an exemplary embodiment may move the plurality of antennas by a predetermined distance in a depthwise direction of the breast 210. In this instance, after the plurality of antennas are moved by the predetermined distance, radio wave sensing may be executed in the same manner as described in the foregoing. For example, in the first through fourth arrangements, radio wave sensing may be executed at a shallowest position in the depthwise direction of the breast 210, and after the radio wave sensing is completed, the plurality of antennas, for example, antenna Nos. 1 through 4, may be moved by the predetermined distance in the depthwise direction of the breast 210. After the plurality of antennas are moved, radio wave sensing may be executed again in the first through fourth arrangements.

Accordingly, the system according to an exemplary embodiment may achieve high precision sensing of a radio wave in diagnostic imaging of breast cancer based on radio wave signal amplitude and phase information obtained in various arrangements in which the plurality of antennas, for example, antenna Nos. 1 through 4, are placed by rotation along the periphery of the breast 210 and movement in the depthwise direction of the breast 210.

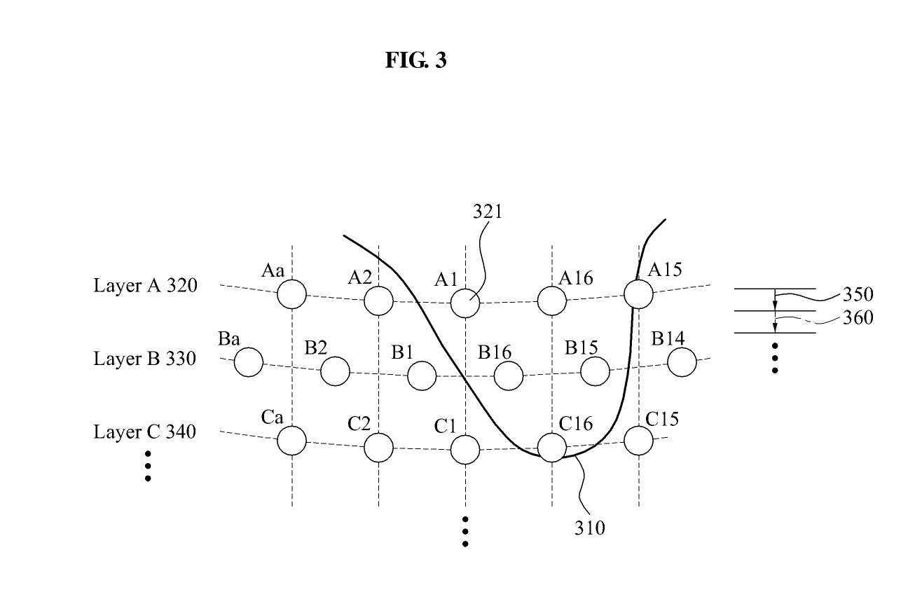

FIG. 3 is a diagram illustrating radio wave sensing in a depthwise direction of a breast in diagnostic imaging of breast cancer according to an exemplary embodiment.

Referring to FIG. 3, radio wave sensing in a depthwise direction of a breast in diagnostic imaging of breast cancer according to an exemplary embodiment may include a breast 310 and a plurality of antennas for each of a plurality of layers, for example, antennas A1, A2, . . . , A16 for a layer A, antennas B1, B2, . . . , B16 for a layer B, and antennas C1, C2, . . . , C16 for a layer C. Here, the plurality of antennas A1, A2, . . . , A16, B1, B2, . . . , B16, C1, C2, . . . , C16 may correspond to transmitting and receiving antennas.

A system according to an exemplary embodiment may arrange the plurality of antennas A1, A2, . . . , A16, B1, B2, . . . , B16, C1, C2, . . . , C16 in the plurality of layers A through C in a depthwise direction of the breast 310. For example, the antennas A1, A2, . . . , A16 may be arranged in the layer A, the antennas B1, B2, . . . , B16 may be arranged in the layer B, and the antennas C1, C2, . . . , C16 may be arranged in the layer C. In this instance, the plurality of antennas A1, A2, . . . , A16, B1, B2, . . . , B16, C1, C2, . . . , C16 for each of the plurality of layers may be arranged in a staggered manner, relative to the antennas A1, A2, . . . , A16, B1, B2, . . . , B16, C1, C2, . . . , C16 of adjacent layers. For example, the antennas B1, B2, . . . , B16 of the layer B may be staggered relative to the antennas A1, A2, . . . , A16 of the layer A and the antennas C1, C2, . . . , C16 of the layer C.

Also, the system according to an exemplary embodiment may set a first antenna among the plurality of antennas A1, A2, . . . , A16, B1, B2, . . . , B16, C1, C2, . . . , C16 for each of the plurality of layers A through C, may transmit a radio wave signal from the first antenna to the remaining antennas, other than the first antenna in the same layer as the first antenna, and may receive a scattered wave signal for the radio wave signal from the remaining antennas, other than the first antenna in the same layer as the first antenna. In this instance, a new first antenna may be set among the plurality of antennas for each of the plurality of layers, and radio wave sensing may be executed iteratively.

For example, when an antenna A1 321 in the layer A is set to be a first antenna, the antenna A1 321 may transmit a radio wave signal to the remaining antennas A2, A3, . . . , A16, other than the antenna A1 321 in the same layer A, and the remaining antennas A2, A3, . . . , A16 may receive a scattered wave signal for the radio wave signal. After the remaining antennas A2, A3, . . . , A16, other than the antenna A1 321 receive the scattered wave signal for the radio wave signal, an antenna A2 may be set to be a new first antenna, and this process may be performed iteratively until the plurality of antennas A1, A2, . . . , A16 arranged in the layer A 320 are all set to be a first antenna. In this instance, radio wave sensing described in the foregoing may be executed again in the layer B 330 and the layer C 340.

After the radio wave sensing is executed for each of the plurality of layers A, B, and C, the system according to an exemplary embodiment may move the plurality of antennas A1, A2, . . . , A16, B1, B2, . . . , B16, C1, C2, . . . , C16 for each of the plurality of layers A through C by a predetermined distance, for example, a distance 350 and a distance 360, in a depthwise direction of the breast 310.

In this instance, after the plurality of antennas A1, A2, . . . , A16, B1, B2, . . . , B16, C1, C2, . . . , C16 are moved by the predetermined distance 350, the system according to an exemplary embodiment may execute the radio wave sensing for each of the plurality of layers. After the radio wave sensing is completed, the system according to an exemplary embodiment may move the plurality of antennas A1, A2, . . . , A16, B1, B2, . . . , B16, C1, C2, . . . , C16 by the predetermined distance 360 and may execute the radio wave sensing again.

Also, the system according to an exemplary embodiment may set a first antenna among the plurality of antennas for preset adjacent layers among the plurality of layers A through C, may transmit a radio wave signal from the first antenna, and may receive a scattered wave signal for the radio wave signal from the remaining antennas, other than the first antenna, among the plurality of antennas for the preset adjacent layers.

For example, when a layer A 320 and a layer B 330 among the plurality of layers A through C are preset to be adjacent layers, an antenna A1 321 may be set to be a first antenna among the plurality of antennas A1, A2, . . . , A16 for the layer A 320 and the plurality of antennas B1, B2, . . . , B16 for the layer B 330, the antenna A1 321 may transmit a radio wave signal to the remaining antennas A2, . . . , A16, and B1, B2, . . . , B16, other than the antenna A1 321, and the remaining antennas A2, . . . , A 16, and B1, B2, . . . , B16 may receive a scattered wave signal for the radio wave signal. In this instance, when a particular antenna among the plurality of antennas for the layer A is set to be a first antenna, this communication process may be executed from the layer A, and when a particular antenna among the plurality of antennas for the layer B is set to be a first antenna, this communication process may be executed from the layer B.

Also, after the remaining antennas A2, . . . , A16, B1, B2, . . . , B16, other than the antenna A1 321 receive the scattered wave signal for the radio wave signal, one of the remaining antennas A2, . . . , A16, and B1, B2, . . . , B16, other than the antenna A1 321 may be set to be a new first antenna, the new first antenna may transmit a radio wave signal, and the remaining antennas, other than the first antenna, may receive a scattered wave signal for the radio wave signal.

The radio wave sensing may be executed iteratively until the plurality of antennas A1, A2, . . . , A16, B1, B2, . . . , B16 for the preset adjacent layers A and B are all set to be a first antenna.

Also, after the radio wave sensing is executed on the preset adjacent layers among the plurality of layers A through C, the system according to an exemplary embodiment may move the plurality of antennas A1, A2, . . . , A16, B1, B2, . . . , B16, C1, C2, . . . , C16 by the predetermined distance, for example, the distance 350 and the distance 360, in the depthwise direction of the breast 310.

In this instance, the system according to an exemplary embodiment may move the plurality of antennas A1, A2, . . . , A16, B1, B2, . . . , B16, C1, C2, . . . , C16 by the predetermined distance 350, execute the radio wave sensing in the adjacent layers, and after the radio wave sensing is completed, move the plurality of antennas A1, A2, . . . , A16, B1, B2, . . . , B16, C1, C2, . . . , C16 by the predetermined distance 360, and execute the radio wave sensing again.

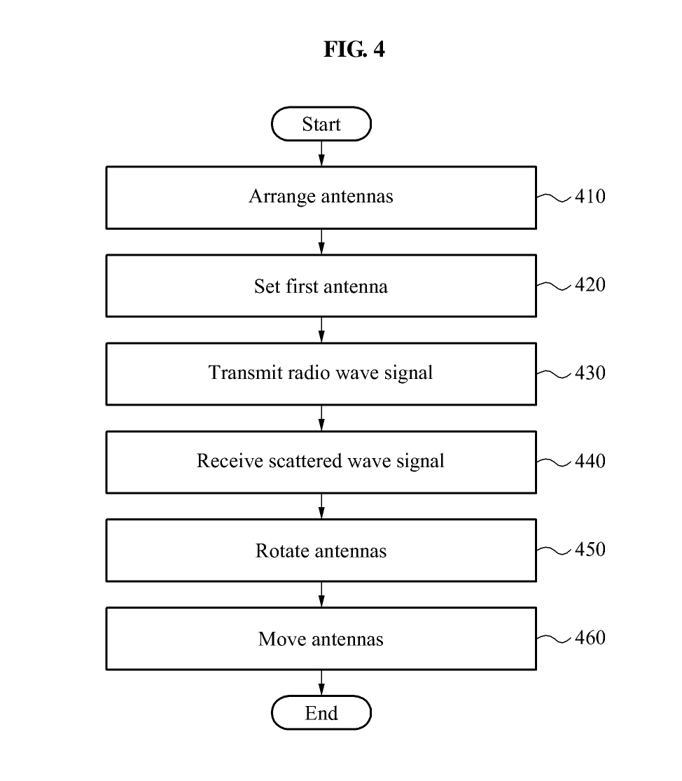

FIG. 4 is a flowchart illustrating a method of sensing a radio wave along a periphery of a breast in diagnostic imaging of breast cancer according to an exemplary embodiment.

Referring to FIG. 4, in operation 410, a plurality of transmitting and receiving antennas may be arranged along a periphery of a breast.

In operation 420, at least one of the plurality of transmitting and receiving antennas may be set to be a first antenna.

In operation 430, the first antenna may transmit a radio wave signal.

In operation 440, the remaining antennas, other than the first antenna, among the plurality of transmitting and receiving antennas may receive a scattered wave signal for the radio wave signal. Here, radio wave signal amplitude and phase information may be obtained from the received radio wave signal.

After the scattered wave signal is received, at least one of the remaining antennas, other than the first antenna transmitting the radio wave signal, may be set to be a new first antenna.

In operation 450, the plurality of transmitting and receiving antennas may be rotated by a predetermined angle. In this instance, the plurality of transmitting and receiving antennas may be re-arranged by being rotated the predetermined angle along the periphery of the breast.

In operation 460, after the plurality of transmitting and receiving antennas are rotated, the plurality of transmitting and receiving antennas may be moved by a predetermined distance in a depthwise direction of the breast.

After, the plurality of transmitting and receiving antennas are moved in the depthwise direction of the breast, radio wave sensing may be executed iteratively at each location to which the plurality of transmitting and receiving antennas are moved in the depthwise direction of the breast.

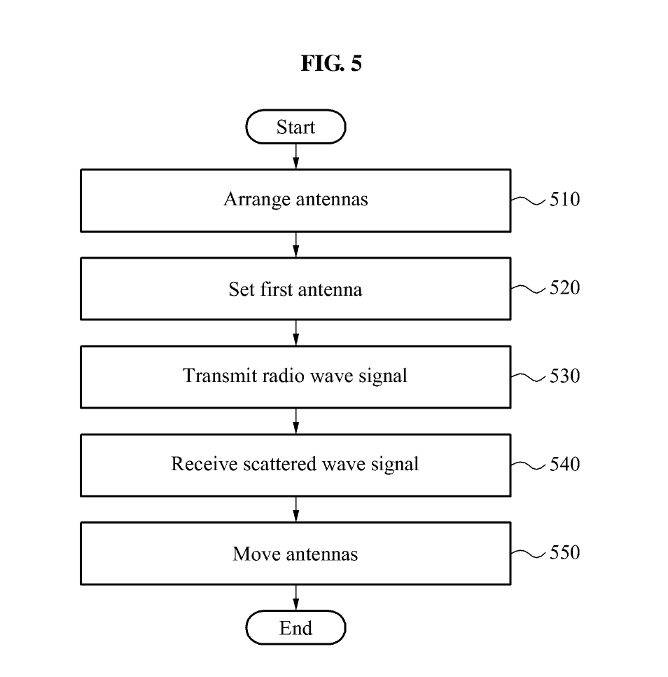

FIG. 5 is a flowchart illustrating a method of sensing a radio wave in a depthwise direction of a breast in diagnostic imaging of breast cancer according to an exemplary embodiment.

Referring to FIG. 5, in operation 510, a plurality of transmitting and receiving antennas may be arranged in each of a plurality of layers in a depthwise direction of a breast. In this instance, the plurality of transmitting and receiving antennas for each of the plurality of layers may be arranged in a staggered manner relative to the transmitting and receiving antennas of adjacent layers.

In operation 520, at least one of the plurality of transmitting and receiving antennas for each of the plurality of layers may be set to be a first antenna.

In operation 530, the first antenna may transmit a radio wave signal.

In operation 540, the remaining antennas, other than the first antenna, among the plurality of transmitting and receiving antennas for each of the plurality of layers may receive a scattered wave signal for the radio wave signal. Here, radio wave signal amplitude and phase information may be obtained from the received radio wave signal.

After the scattered wave signal is received for each of the plurality of layers, at least one of the remaining antennas, other than the first antenna transmitting the radio wave signal, may be set to be a new first antenna.

In operation 550, the plurality of transmitting and receiving antennas for each of the plurality of layers may be moved by a predetermined distance in a depthwise direction of the breast.

After the plurality of transmitting and receiving antennas are moved in the depthwise direction of the breast, radio wave sensing may be executed iteratively at each location to which the plurality of transmitting and receiving antennas are moved in the depthwise direction of the breast.

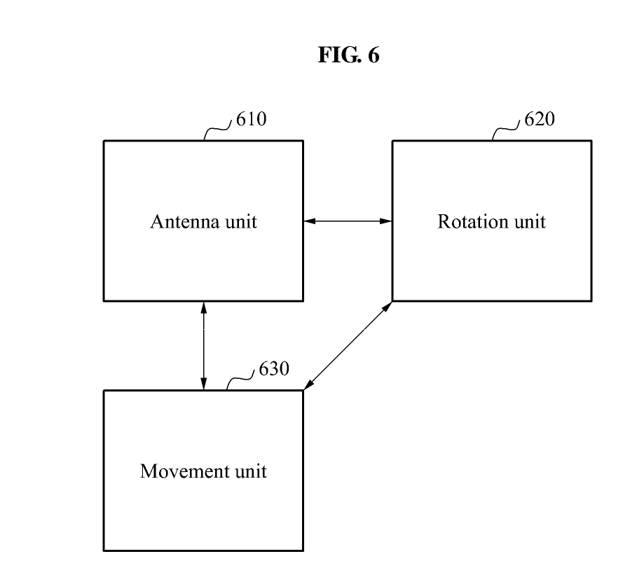

FIG. 6 is a block diagram illustrating a system for sensing a radio wave along a periphery of a breast in diagnostic imaging of breast cancer according to an exemplary embodiment.

Referring to FIG. 6, the system for sensing a radio wave along a periphery of a breast in diagnostic imaging of breast cancer may include an antenna unit 610, a rotation unit 620, and a movement unit 630.

The antenna unit 610 may include a plurality of transmitting and receiving antennas that may be arranged along a periphery of a breast. Among the plurality of transmitting and receiving antennas, at least one first antenna may transmit a radio wave signal, and the remaining antennas, other than the first antenna, may receive a scattered wave signal for the radio wave signal.

After the scattered radio signal is received, at least one new first antenna may be set among the remaining antennas, other than the first antenna transmitting the radio wave signal.

In this instance, radio wave signal amplitude and phase information may be obtained from the received radio wave signal.

The rotation unit 620 may rotate the plurality of transmitting and receiving antennas by a predetermined angle. The rotation unit 620 may re-arrange the plurality of transmitting and receiving antennas by rotating the plurality of transmitting and receiving antennas by the predetermined angle along the periphery of the breast.

The movement unit 630 may move the plurality of transmitting and receiving antennas by a predetermined distance in a depthwise direction of the breast after the plurality of transmitting and receiving antennas are rotated.

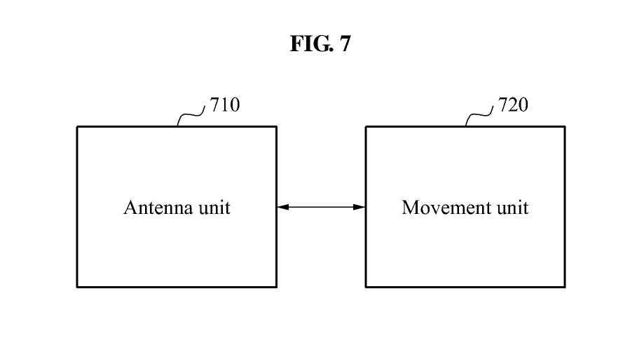

FIG. 7 is a block diagram illustrating a system for sensing a radio wave in a depthwise direction of a breast in diagnostic imaging of breast cancer according to an exemplary embodiment.

Referring to FIG. 7, the system for sensing a radio wave in a depthwise direction of a breast in diagnostic imaging of breast cancer may include an antenna unit 710 and a movement unit 720.

The antenna unit 710 may include a plurality of transmitting and receiving antennas for each of a plurality of layers. The plurality of transmitting and receiving antennas may be arranged along a periphery of a breast in each of the plurality of layers in a depthwise direction of the breast. Among the plurality of transmitting and receiving antennas for each of the plurality of layers, at least one first antenna may transmit a radio wave signal, and the remaining antennas, other than the first antenna, among the plurality of transmitting and receiving antennas for each of the plurality of layers may receive a scattered wave signal for the radio wave signal.

The plurality of transmitting and receiving antennas for each of the plurality of layers may be arranged in a staggered manner, relative to the transmitting and receiving antennas of adjacent layers. After the scattered wave signal is received for each of the plurality of layers, at least one new first antenna may be set among the remaining antennas, other than the first antenna transmitting the radio wave signal.

Among the plurality of transmitting and receiving antennas for preset adjacent layers among the plurality of layers, at least one first antenna may transmit a radio wave signal, and the remaining antennas, other than the first antenna, among the plurality of transmitting and receiving antennas for the preset adjacent layers may receive a scattered wave signal for the radio wave signal. After the scatter wave signal is received in the adjacent layers, at least one new first antenna may be set among the remaining antennas, other than the first antenna transmitting the radio wave signal.

The movement unit 720 may move the plurality of transmitting and receiving antennas for each of the plurality of layers by a predetermined distance in a depthwise direction of the breast.

The units described herein may be implemented using hardware components, software components, or a combination thereof. For example, a processing device may be implemented using one or more general-purpose or special purpose computers, such as, for example, a processor, a controller and an arithmetic logic unit, a digital signal processor, a microcomputer, a field programmable array, a programmable logic unit, a microprocessor or any other device capable of responding to and executing instructions in a defined manner. The processing device may run an operating system (OS) and one or more software applications that run on the OS. The processing device also may access, store, manipulate, process, and create data in response to execution of the software. For purpose of simplicity, the description of a processing device is used as singular; however, one skilled in the art will appreciated that a processing device may include multiple processing elements and multiple types of processing elements. For example, a processing device may include multiple processors or a processor and a controller. In addition, different processing configurations are possible, such as parallel processors.

The software may include a computer program, a piece of code, an instruction, or some combination thereof, for independently or collectively instructing or configuring the processing device to operate as desired. Software and data may be embodied permanently or temporarily in any type of machine, component, physical or virtual equipment, computer storage medium or device, or in a propagated signal wave capable of providing instructions or data to or being interpreted by the processing device. The software also may be distributed over network coupled computer systems so that the software is stored and executed in a distributed fashion. In particular, the software and data may be stored by one or more computer readable recording mediums.

The computer readable recording medium may include any data storage device that can store data which can be thereafter read by a computer system or processing device. Examples of the computer readable recording medium include read-only memory (ROM), random-access memory (RAM), CD-ROMs, magnetic tapes, floppy disks, optical data storage devices. Also, functional programs, codes, and code segments for accomplishing the example embodiments disclosed herein can be easily construed by programmers skilled in the art to which the embodiments pertain based on and using the flow diagrams and block diagrams of the figures and their corresponding descriptions as provided herein.

According to exemplary embodiments, there may be provided, in diagnostic imaging of breast cancer, a method, apparatus, and system for high precision sensing of a radio wave along a periphery of a breast using a plurality of transmitting and receiving antennas.

According to exemplary embodiments, there may be provided, in diagnostic imaging of breast cancer, a method, apparatus, and system for high precision sensing of a radio wave by rotating and moving a plurality of transmitting and receiving antennas in the process of using the plurality of transmitting and receiving antennas.

According to exemplary embodiments, there may be provided, in diagnostic imaging of breast cancer, a method, apparatus, and system for high precision sensing of a radio wave in a depthwise direction of a breast using a plurality of transmitting and receiving antennas for a plurality of layers.

According to exemplary embodiments, there may be provided a method, apparatus, and system for arranging a plurality of transmitting and receiving antennas in each of a plurality of layers of a breast in the process of using the plurality of transmitting and receiving antennas for the plurality of layers.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. A method of sensing a radio wave in diagnostic imaging of breast cancer, the method comprising:
arranging a plurality of transmitting and receiving antennas along a periphery of a breast;
transmitting a radio wave signal from at least one first antenna among the plurality of transmitting and receiving antennas;
receiving a scattered wave signal for the radio wave signal from the remaining antennas, other than the first antenna, among the plurality of transmitting and receiving antennas; and
rotating the plurality of transmitting and receiving antennas by a predetermined angle.

2. The method of claim 1, wherein the rotating of the plurality of transmitting and receiving antennas comprises re-arranging the plurality of transmitting and receiving antennas by rotating the plurality of transmitting and receiving antennas by the predetermined angle along the periphery of the breast.

3. The method of claim 1, further comprising:
moving the plurality of transmitting and receiving antennas by a predetermined distance in a depthwise direction of the breast after rotating the plurality of transmitting and receiving antennas.

4. The method of claim 1, further comprising:
setting at least one new first antenna among the remaining antennas, other than the first antenna transmitting the radio wave signal, after receiving the scattered wave signal.

5. The method of claim 1, wherein the receiving of the scattered wave signal for the radio wave signal comprises obtaining radio wave signal amplitude and phase information from the received radio wave signal.

6. A method of sensing a radio wave in diagnostic imaging of breast cancer, the method comprising:
arranging a plurality of transmitting and receiving antennas along a periphery of the breast in a plurality of layers in a depthwise direction of a breast;
transmitting a radio wave signal from at least one first antenna among the plurality of transmitting and receiving antennas for the plurality of layers;
receiving a scattered wave signal for the radio wave signal from the remaining antennas, other than the first antenna, among the plurality of transmitting and receiving antennas for the plurality of layers; and
moving the plurality of transmitting and receiving antennas for the plurality of layers by a predetermined distance in the depthwise direction of the breast.

7. The method of claim 6, wherein the arranging of the plurality of transmitting and receiving antennas in the plurality of layers comprises arranging the plurality of transmitting and receiving antennas in the plurality of layers in a staggered manner, relative to the transmitting and receiving antennas of adjacent layers.

8. The method of claim 6, further comprising:
setting at least one new first antenna among the remaining antennas, other than the first antenna transmitting the radio wave signal, after receiving the scattered wave signal in the plurality of layers.

9. The method of claim 6, further comprising:
transmitting the radio wave signal from the at least one first antenna among the plurality of transmitting and receiving antennas for preset adjacent layers among the plurality of layers; and
receiving the scattered wave signal for the radio wave signal from the remaining antennas, other than the first antenna, among the plurality of transmitting and receiving antennas for the preset adjacent layers.

10. The method of claim 9, further comprising:
setting at least one new first antenna among the remaining antennas, other than the first antenna transmitting the radio wave signal, after receiving the scattered wave signal in the preset adjacent layers.

11. A system for sensing a radio wave in diagnostic imaging of breast cancer, the system comprising:
a plurality of transmitting and receiving antennas;
a rotation unit; and
a movement unit,
wherein the plurality of transmitting and receiving antennas are arranged along a periphery of a breast, at least one first antenna among the plurality of transmitting and receiving antennas transmits a radio wave signal, and the remaining antennas, other than the first antenna, receive a scattered wave signal for the radio wave signal, and
the rotation unit rotates the plurality of transmitting and receiving antennas by a predetermined angle, and re-arranges the plurality of transmitting and receiving antennas by rotating the plurality of transmitting and receiving antennas by the predetermined angle along the periphery of the breast.

12. The system of claim 11, wherein the movement unit moves the plurality of transmitting and receiving antennas by a predetermined distance in a depthwise direction of the breast after the plurality of transmitting and receiving antennas are rotated, and
after the scattered wave signal is received from the remaining antennas, other than the first antenna, at least one new first antenna is set among the remaining antennas other than the first antenna transmitting the radio wave signal.

13. A system for sensing a radio wave in diagnostic imaging of breast cancer, the system comprising:
a plurality of transmitting and receiving antennas for a plurality of layers; and
a movement unit,
wherein the plurality of transmitting and receiving antennas for the plurality of layers are arranged along a periphery of a breast in the plurality of layers in a depthwise direction of the breast, at least one first antenna among the plurality of transmitting and receiving antennas for the plurality of layers transmits a radio wave signal, and the remaining antennas, other than the first antenna, among the plurality of transmitting and receiving antennas for the plurality of layers receive a scattered wave signal for the radio wave signal, and
the movement unit moves the plurality of transmitting and receiving antennas for the plurality of layers by a predetermined distance in the depthwise direction of the breast.

14. The system of claim 13, wherein the plurality of transmitting and receiving antennas for the plurality of layers are arranged in a staggered manner relative to the transmitting and receiving antennas of adjacent layers, and after the scattered wave signal is received from the remaining antennas, other than the first antenna, among the plurality of transmitting and receiving antennas for the plurality of layers, at least one new first antenna is set among the remaining antennas other than the first antenna transmitting the radio wave signal.

15. The system of claim 13, wherein the at least one first antenna among the plurality of transmitting and receiving antennas for preset adjacent layers among the plurality of layers transmit the radio wave signal, and the remaining antennas, other than the first antenna, among the plurality of transmitting and receiving antennas for the preset adjacent layers receive a scattered wave signal for the radio wave signal.
